(19) United States
(12) Patent Application Publication
(10) Pub. No.: US 2007/0275458 A1
(43) Pub. Date: Nov. 29, 2007

(54) Three dimensional-BIO-mimicking active scaffolds

(75) Inventor: Pelagia-Irene Gouma, Port Jefferson, NY (US)

Correspondence Address: DILWORTH & BARRESE, LLP, 333 EARLE OVINGTON BLVD., SUITE 702, UNIONDALE, NY, 11553, US

(73) Assignee: The Research Foundation of State University of New York, Albany, NY (US)

(21) Appl. No.: 11/634,590

(22) Filed: Dec. 6, 2006

Related U.S. Application Data
(60) Provisional application No. 60/748,924, filed on Dec. 9, 2005.

(51) Int. Cl. C12M 1/14

(52) U.S. Cl. 435/297.4; 977/923

(57) ABSTRACT

The invention provides a Cellulose acetate (CA) thin, porous membranes produced by electrospinning precursor polymer solutions in acetone at room temperature and a process for manufacturing the same. The invention also provides a Cellulose acetate (CA) thin, porous membranes produced by electrospinning precursor polymer solutions in acetone at room temperature further comprising ceramic nano-structured component (carbon nanotubes) in the polymer membranes to provide additional strength and porosity and a process for manufacturing the same. The fabricated CA-CT membranes specifically mimic the topography and porosity of natural Extracellular Matrix (ECM) and can be used as scaffolding for cell growth.

### CROSS REFERENCE TO RELATED APPLICATION

This application claims priority to a provisional application filed in the United States Patent and Trademark Office on Dec. 9, 2005 and assigned Ser. No. 60/748,924, the contents of which are incorporated herein in its entirety by reference.

### FIELD OF THE INVENTION

The present invention relates to the field of tissue engineering. In particular, the present invention relates to novel nanofiber scaffolds and the electrospinning process used to produce the scaffolds of the present invention using different polymer compositions.

### BACKGROUND OF THE INVENTION

The repair and replacement of diseased tissue structures and organs requires an enormous expenditure of health-care resources. For example, approximately 500,000 coronary artery bypass surgeries are performed each year in the United States.

Tissue engineering aims at restoring, maintaining or improving tissue function so as to extend and/or preserve the well being of an individual while decreasing the major cost burden on the medical community. [1-3]. These natural processes are occurring in nature using the 3D-structure of ECM (the natural scaffold), which allows cells to grow, proliferate and differentiate within it [2,4]. Artificial scaffolds have been made and used for therapeutic purposes (i.e. cardiac or skin implants) from natural polymers that desorb or degrade within the body [5].

The major challenge for tissue engineering researchers is to find materials and processing techniques that allow them to produce ECM mimicking scaffolds that promote cell growth and organization into a specific architecture, inducing differentiated cell function [2]. ECM is a complex three-dimensional ultrastructure of proteins, proteoglycans and glycoproteins, used for cells growth in native tissue [6]. In fact, there are many different types of ECMs for different parts of the body, for example, fibrous proteins are dominant material in tendon, polysaccharides are found largely existing in cartilage and so the forth. Collagens have been found to be the key proteins in ECM and also are the most ample proteins in the whole body [6].

ECM provides attachment sites and mechanical support for cells [1]. The topology of ECM has been found to affect the cell structure, functionality and its physiological responsiveness [2]. The geometry of the natural matrix was reported to modulate the cell polarity [2]. Thyroid cells, smooth muscle cell and hepatocytes are different types of cells found to be affected by ECM's topology, with 3D-structures inducing cell differentiation more effectively than 2D configurations [2]. The arrangement of ECM's configuration involves multiple length scales, layers and morphologies [5]. However, although much is know about 3-D scaffolding of materials according to ECM topology to proliferate cell growth, satisfactory techniques and/or synthetic scaffolds have not been easily to construct.

Accordingly, there is a need for bioengineered tissue substitutes, such as endothelial cell substitutes, that can be custom-engineered to match the biomechanical, biochemical, and biological needs of the specific tissue or organ they are designed to replace.

One object of the present invention is to provide a method of producing an ECM comprising Cellulose acetate that mimics the topology and porosity of ECM.

Another object of the invention is to provide an electrospinning process for making an artificial ECM comprising Cellulose acetate that mimics the topology and porosity of natural ECM.

Still another object of the invention is to provide a method of producing an artificial ECM comprising Cellulose acetate and carbon nanotubes that mimics the topology and porosity of natural ECM.

The aforementioned objects as well as others are further described below and overcome the shortcomings of the prior art described above.

### BRIEF DESCRIPTION OF THE INVENTION

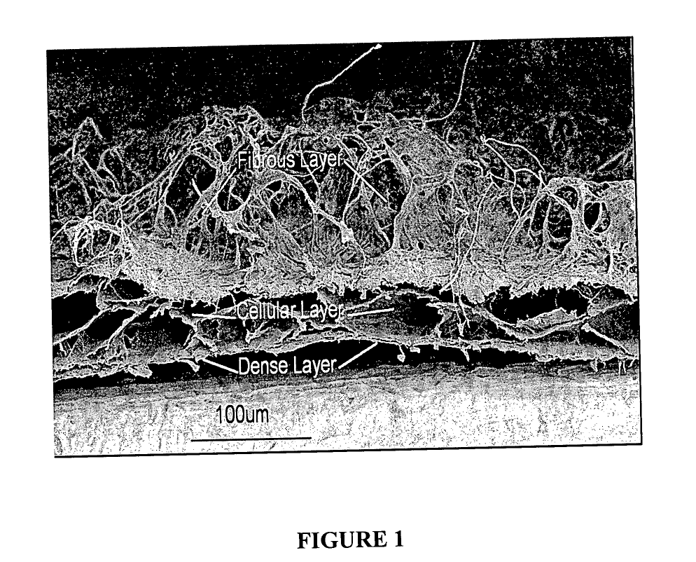

FIG. 1 shows a scanning electron micrograph of the cross sectional as view of the UBM specimen.

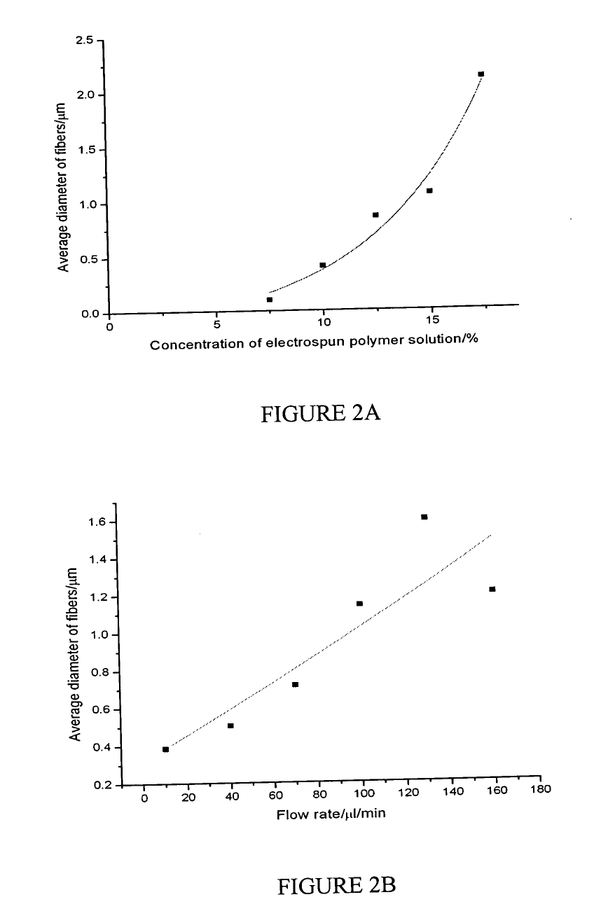

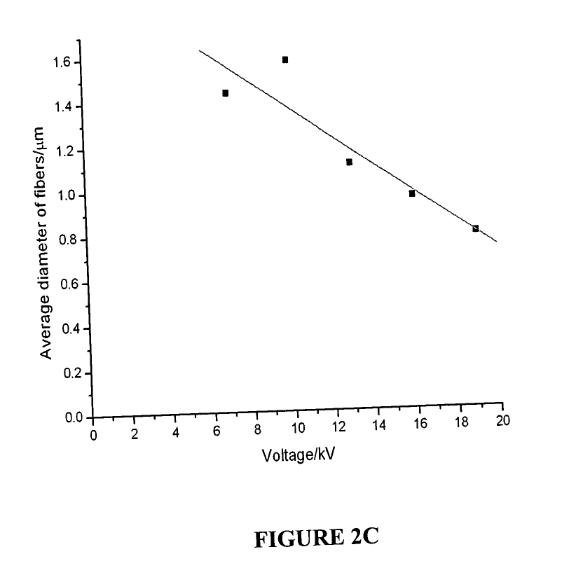

FIG. 2 shows the measured effect of the process parameters on the fiber structures.

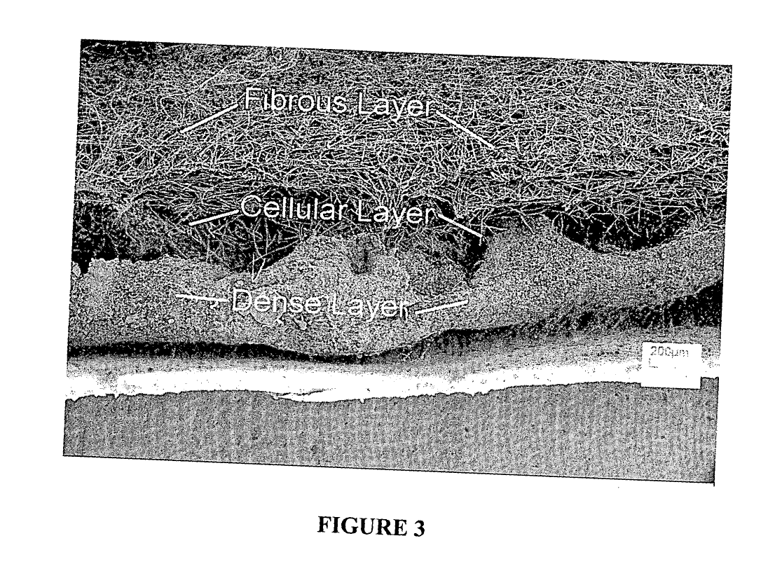

FIG. 3 shows the SEM image of this designed UBM-mimicking scaffold.

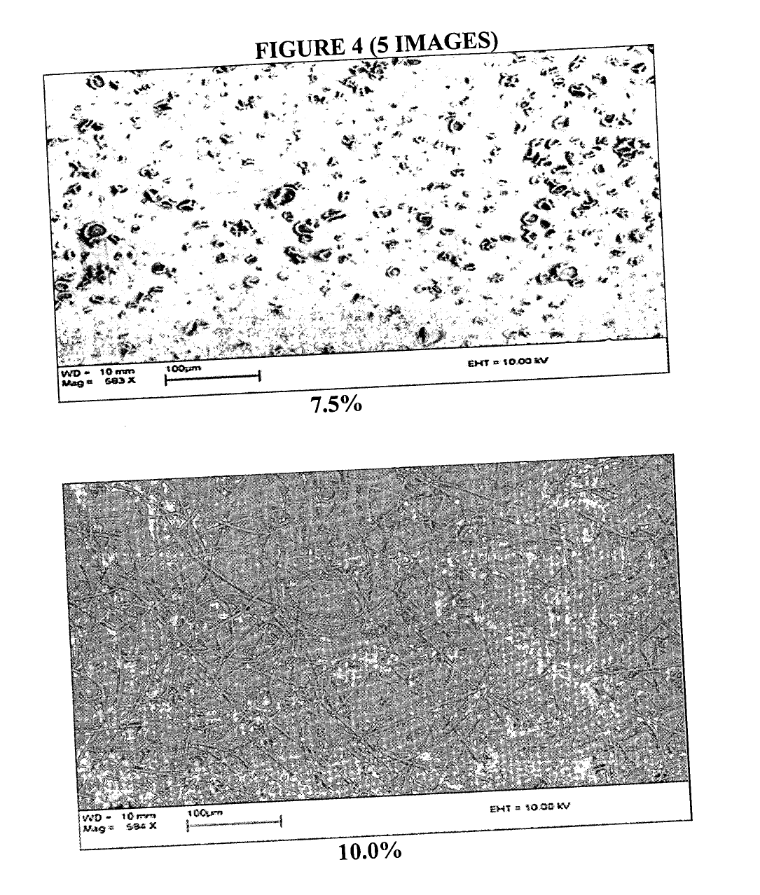

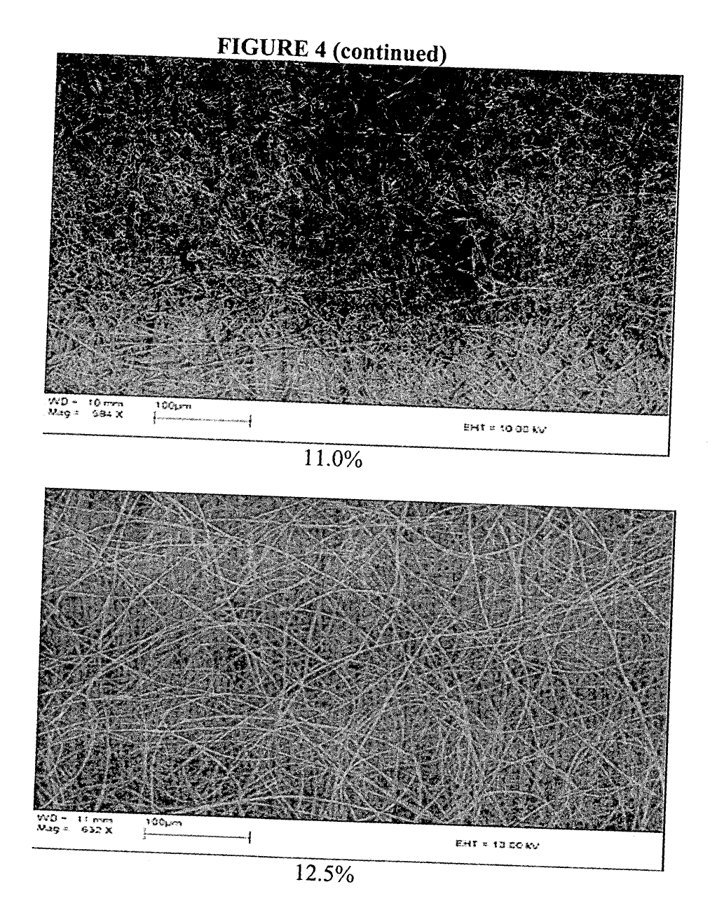

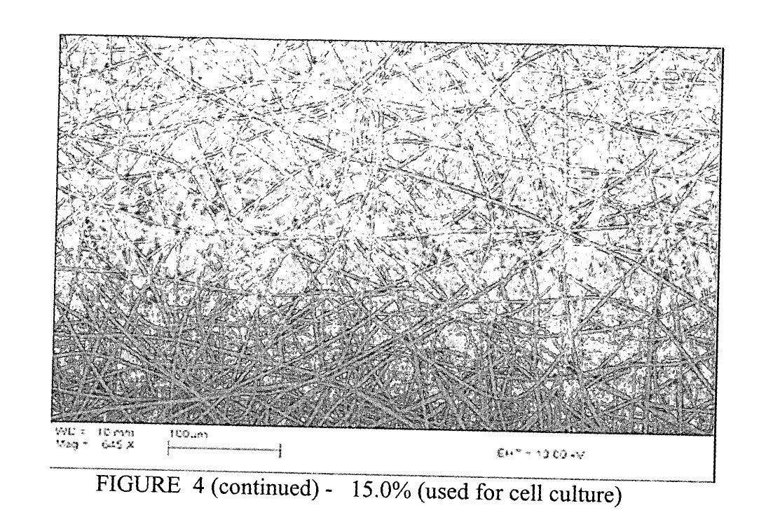

FIG. 4 shows the SEM images of different compositions of CA solutions.

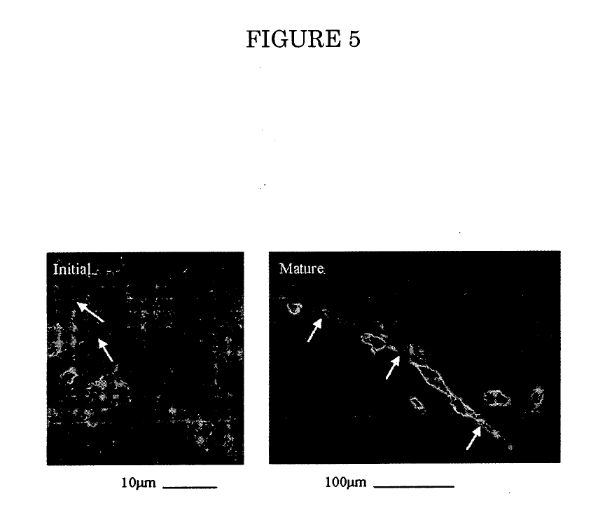

FIG. 5 shows a Human umbilical vein endothelial cells growing on CA+CT membranes.

### SUMMARY OF THE INVENTION

The present invention is directed to an extracellular matrix comprising a three-dimensional non-woven scaffold. The three-dimensional non-woven scaffold comprises one or more layers of one or more arrays of microfibers, wherein one or more of the arrays of microfibers is arranged so as to mimic a configuration of natural extracellular matrix (ECM).

In one embodiment of the invention, the extracellular matrix is produced by an electrospinning process. In another embodiment of the invention, the one or more arrays of microfibers contain cellulose acetate (CA). The extracellular matrix comprising (CA) may also contain carbon nanotubes.

The present invention is also directed to a method for synthesizing an extracellular matrix comprising a three-dimensional non-woven scaffold wherein the method comprises: (a) dissolving at least one polymer in a biocompatible solvent to produce an electrospinning polymer solution and (b) subjecting the electrospinning polymer solution of step (a) to an electric field between about 5 and about 20 kV at a flow rate between about 10 l/min and about 200 l/min to produce single layer mats of variable morphologies.

The electrospinning process of the present invention uses dry deposition conditions and therefore only solutes can be finally deposited on the collector due to the evaporation of the solvent during the ejection process. By means of controlling the voltage of the electric field, the flow rate of the polymer solution, the distance between the capillary and the collector and the concentration of the polymer solution, the diameters of nanofibers can be tailored easily.

One advantage of the present invention is that the extracellular matrix of the present invention closely mimics the target tissues of that they are designed after and can be used to produce those functional tissues. Another advantage of the present invention is that the electrospinning method of the present invention is a one-step electrospinning technique and therefore is easy to use and is cost effective.

These and other aspects and advantages are further discussed in the detailed description and the examples below.

### DETAILED DESCRIPTION OF THE INVENTION

The present invention is directed to an extracellular matrix comprising a three-dimensional non-woven scaffold wherein the scaffold comprises one or more layers of one or more arrays of microfibers. The one or more of the arrays of microfibers is arranged so as to mimic a configuration of natural extracellular matrix (ECM). The ECM is also referred to as the natural scaffold which when combined with a cell culture and/or cell-culturing agents provides the structure for new cell growth.

In one embodiment of the invention, the extracellular matrix described above is produced using an electrospinning process. The electrospinning process is simple, cost effective and produces three-dimensional structures much like those produced in nature. The electrospinning process is further described below and in the examples. In another embodiment of the invention, the polymer used to produce the artificial ECM is cellulose acetate. This material is plentiful in nature and is therefore readably available and inexpensive to use. The characteristics of this material are favorable for the electrospinning process. The ECM may also contain carbon nanotubes (CT), which provides additional structure and a porous arrangement so as to allow permeability of fluids and gases much like a natural ECM.

The extracellular matrix of any of embodiments described above can contain multiple microfibers having a diameter that ranges from about 0.5 μm to about 100 μm, preferably between about 10 μm and 100 μm, and more preferably between about 30 μm and about 70 μm.

In order to facilitate cell growth on the ECM of the present invention, the ECM may be coated with one or more cell adhesion-enhancing agents. These agents include but are not limited collagen, laminin, and fibronectin. The ECM may also contain cells cultured on the scaffold to form a target tissue substitute. The target tissue that may be formed using the ECM of the present invention may be an arterial blood vessel, wherein an array of microfibers is arranged to mimic the configuration of elastin in the medial layer of an arterial blood vessel. In the alternative, other cells may be cultured on the ECM of the present invention. These cells include, but are not limited to, cells cultured on the scaffold to form a blood vessel substitute, epithelial cells cultured on the scaffold to form epithelial tissue, muscle cells cultured on the scaffold to form muscle tissue, endothelial cells cultured on the scaffold to form endothelial tissue, skeletal muscle cells cultured on the scaffold to form skeletal muscle tissue, cardiac muscle cells cultured on the scaffold to form cardiac muscle tissue, collagen fibers cultured on the scaffold to form cartilage, interstitial valvular cells cultured on the scaffold to form valvular tissue and mixtures thereof.

In one embodiment of the invention, the extracellular matrix of the present invention comprises at least three layers of microfibers, wherein a first array of microfibers is arranged to mimic a dense layer of the endothelia tissue, wherein a second array of microfibers is arranged to mimic a cellular layer of the endothelia tissue, and wherein a third layer of microfibers is arranged to mimic a fibrous layer of the endothelia tissue. This type of arrangement can be used to mimic a urinary bladder matrix (UBM) and may contain a keratinocyte growth substrate.

One type of ECM obtained from porcine urinary bladder matrix (UBM) has found to have potential use in keratinocyte growth substrate [7]. FIG. 1 shows a scanning electron micrograph of the cross sectional as view of the UBM specimen. A dense basal layer, followed by a multilayered (3-layer) cellular-type structure with flattened, elongated, ellipsoidal-shaped pockets, topped by a non-uniformly, loosely shaped fibrous layer are observed. Table 1 summarizes the dimensions of the key features of the UBM's architecture.

The structure of the fibrous layer consists of uniform size fibers with an average diameter of 1.7±0.1 μm. The cellular-type layers vary widely having pores that range from about 11.8 μm to about 72 μm with an average pore size estimated to be 36.6 μm. The dense bottom level has a thickness of 2.0 μm. Structures with the same shape and appearance can be produced using the above process these structures mimic the bioactivity of the UBM.

As mentioned above, the present invention is also directed to a method for synthesizing an extracellular matrix comprising a three-dimensional non-woven scaffold. The method comprises: (a) dissolving at least one polymer with a biocompatible solvent to produce an electrospinning polymer solution; (b) subjecting the electrospinning polymer solution of step (a) to an electric field between about 5 and about 20 kV at a flow rate between about 10 l/min and about 200 l/min to produce single layer mats of variable morphologies.

The method described above for synthesizing an extracellular matrix of the present invention may use cellulose acetate (CA) as the polymer and/or may include carbon nanotubes (CT) as well. In one embodiment of the invention the above described process is used to synthesize an extracellular matrix having at least three single layer mats of variable morphologies that when produced mimics the configuration of one or more structural elements in a target tissue. In order to facilitated the proper spacing and pore size when using the electrospinning process of the present invention to produce a multilayer structure, a removable copper wire digitated-shaped template mimicking the desired structure may be inserted between at least two of the three single layer mats so as to induce the electrospinning process to produce a layer that mimics the copper wire template. Once the electrospinning process is completed, the copper wire template can be removed sterilized and reused.

In order to illustrate various illustrative embodiments of the present inventions, the following examples are provided.

### EXAMPLES

### Electrospun Materials Using Cellulose Acetate

Materials & Methods

Processing of Cellulose Acetate

CA (29,000 g/mol) and 40% substitution (acetyl groups) purchased from Fluka (Fluka Chemie GmbH CH-9471 Buchs) and acetone with compositions ranging from 7.5 to 17.5% w/v were made simply by mixing CA powder and acetone at room temperature. Magnetic stirring was applied to the solutions due to precipitation of CA.

Electrospinning Conditions

The apparatus of electrospinning setup consists of high voltage power supply which could provide up to several tens of Kilovolts, say 40 kV, with two electrodes, metering pump, a glass syringe with a small diameter needle (millimeter scale) and collector usually a metal screen. One of the two electrodes was attached to the needle of the syringe while the other was attached to the collector. In the initial experiments, the flow rate was varied from 10 to 160 μl/min and the strength of electric field was varied from 7 to 19 kV so as to produce single layer mats of variable morphologies deposited on aluminum foil.

Characterization

Before examining them under the SEM, the specimens were gold coated for 15 seconds. Scanning electron microscopy (SEM) was performed on a LEO 1550 electron microscope. The average pore size was calculated by adding the height and length divided by 2.

Cellulose Acetate Electrospun Scaffolds

As the most plentiful organic compound existing in the world, cellulose is the primary component of the higher plants' cell walls. The structure of cellulose can be identified as a long chain polymer, which is a long polysaccharide [8]. CA is one of the cellulose derivatives. It can be dissolved in organic solvents such as acetone and acetic acid. CA has very low water solubility, which is an advantage when using it for scaffold fabrication [9]. CA scaffolds used for cardiac cell growth were found to boost the cell growth and helped increase their connectivity. The cell adhesion properties were better than that of other polymeric artificial scaffolds [9]. CA has also been reported to have good biocompatibility [10]. CA scaffolds can be prepared by the electrospinning of the present invention.

Electrospinning can generate CA polymeric fibers with a diameter ranging from a few nanometers to several tens of micrometers. The structures produced are porous. By applying an electric field that is strong enough to overcome the surface tension of the droplet to a syringe containing a polymeric melt or CA containing solution produces, a continuous jet at the tip of the needle. The jet formed moves is formed moving towards a metal-grounded collector placed not far away and results in continuous fiber formation in non-woven mats. By varying the polymer concentration (surface tension) and processing parameters including the strength of electric field, tip-target distance and the flow rate of polymeric solution or melt, the morphology of the mats can be easily modified in a continuous manner, as described below [11]-[14]. The measured effect of the process parameters on the fiber structures is summarized in FIG. 2. When the polymer entanglement is not sufficient resulting in the instability of the polymer solution jet, polymer fibers with beads can be observed.

Solution viscosity and net charge density are two major factors for forming beads [15]. The largest fiber diameters can be obtained for high concentrations of the polymer solution (17.5% w/v), high flow rate (160 μl/min) and low strength of electric field (7 kV). At very low solution concentrations, 7.5% w/v, droplets of polymers were generated and beads were seen under high magnification of SEM. The porosity of such obtained mats is very low and dense. When the concentration went up to 17.5% w/v, the morphology of the mats was open rather than compact as observed for the low concentration polymer solution. Although mechanical tests were not performed on the respective structures, the strengths of two different mats are apparently different: dense beads-like mats are much stronger than loose fiber mats. In order to produce the structure of UBM scaffolds, different CA solutions and processing parameters were designed, according to the knowledge obtained from the preliminary experiments with the single layer mats. In order to mimic the bottom layer of ECM of the present invention, the structure of the mat should be very dense and flat. From the previous experience, low concentration of polymer solution, low flow rate and high electric field will result in dense structure of mat. The solution with concentration of 7.5% w/v was first used as electrospun material to produce the bottom layer one under 30 μl/min and 19 kV.

Considering the middle layers and the bottom layer together, it appeared that the two layers were connected at regularly spaced intervals, separated by large pores in between. Therefore, in order to mimic this, the structure of middle layer needs to be designed between that of a dense and a loose structure. Before electrospinning the middle layer, a copper wire pattern was created to introduce porosity between the first and second layers. Copper wire coils with a diameter of 300 μm were used to make a digitated-shaped template with a separation distance of about 1 mm between two adjacent coils. This template served as a physical separator to induce the channeled (cellular-like) configuration. This removable copper wire template did not interrupt the continuity of the electrospinning process.

The solution at concentration of 10.0% w/v was then electrospun onto the initial layer under the same conditions. The top layer of ECM consists of randomly oriented fibers with large porosity. The processing condition selected for the top layer were 17.5% w/v polymer concentration under 160 μl/min and 7 kV.

Morpholopy of the Bio-Mimiciny Scaffolds

FIG. 3 shows the SEM image of this designed UBM-mimicking scaffold. Although only one middle layer is shown here, the structural features are consistent with the UBM features described above. The size of the structure of the fibrous layer: average diameter of fibers is 2.0±0.5 μm; the cellular layer includes ellipsoidal-shaped pores with average pore size 357.2 μm (closely related to the copper coil size used for patterning purposes), the dense layer's morphology is identical to that of the respective layer of the UBM.

It has been found in studies with ECM scaffolds and hepatocytes that sandwiched configurations (like the cellular one achieved herein) reduce cell spreading and enhance the expression of differentiated cell function [2]. These features make them very promising candidates for the next generation of artificial scaffolds for tissue and organ growth. The fact that such structures may be fabricated in a single step process using minimal patterning that does not disrupt the manufacturing process is an innovation that will be welcomed by the biomaterials community.

As discussed above, the electrospinning process of the present invention is capable of fabricating multilayered structure polymer-based membrane scaffolds that are inspired by the natural ECM's architecture. The potential of the 3D architectures to control cell differentiation through topological control enables the growth of tissue and organs that closely mimic nature. One advantage of the process of the present invention is that all of the above functionality can be obtained using the single step process described immediately above.

TABLE 1
Dimension detail of model ECM

| | Fibrous Layer | Cellular Layer | Dense Layer |
|---|---|---|---|
| Dimension | Fiber Diameter | Average pore | Thickness: |
| | 1.7 ± 0.1 μm | size: 36.6 μm | 2.0 μm |
| | | Average pore | |
| | | height 16.4 ± 7 μm | |
| | | Average pore | |
| | | length 68.3 ± 5 μm | |

Carbon nanotubes may be added to the CA to produce ECM comprising CA-CT mixtures. The process used to produce the ECM is similar to the one described immediately above and is further described below.

Electrospun Materials Using Cellulose Acetate (Ca) and Carbon Nanotubes (CT).

Cellulose Acetate with a number average molecular weight of 29,000 g/mol was purchased from Fluka (Fluka Chemie GmbH CH-9471 Buchs). Acetone, ACS, 99.5+% (Assay) was obtained from Alfa Aesar, Mass., USA. Those two were used for preparing the electrospinnable polymer solutions. As-prepared single walled carbon nanotube (AP-SWNT) was obtained from Carbon Solutions Inc., CA, USA, and used as a secondary ceramic nanoparticles in the polymer membranes for strength. Solutions of CA and acetone with compositions ranging from 7.5 to 20.0% w/v were prepared simply by mixing them together at room temperature (ca. 22-25° C.). The polymer dissolved completely in acetone usually after 2.5 h without stirring. Cellulose Acetate-Carbon Nanotube (CA-CT) solution was prepared by directly adding the nanotube to 15.0% w/v CA acetone solution. In order to obtain homogeneous solution, the CA-CT solution was made under stirring. Precipitation of nanotubes occurs after 5 min. Therefore, the electrospinning process of CA-CT solution was done within 5 min after the completion of solution preparation.

Different compositions of CA solutions were electrospun under a voltage as high as 12 kV with the distance between the syringe and the collector being ca. 15 cm. The flow rate was controlled at 100 μl/min. CA-CT solution was electrospun under the same condition.

Endothelial Cell Culture.

Human umbilical vein endothelial cells were obtained as first passage cultures. Cells were incubated at 37° in a 5% CO₂ humidified atmosphere. The cell culture media was composed of McCoy's SA base media with 20% calf serum, 100 μg/ml heparin, 50 μg/ml endothelial cell growth supplement, 2 mM L-glutamine, without antibiotics (Sigma Chemical Co.) [20]. Coverslips and membranes were sterilized by uv light exposure.

Three types of electrospun materials were tested for their effect on cellular viability:CA alone, A+0.25% CNT, CA+0.5% CNT. Cells were trypsin digested to transfer onto 1 cm² area membrane at a dilution of 1:1 or 1:2. On days 3-5 post seeding, cellular viability was assessed in two ways; cells were either loosely attached and could be washed off by rinsing the material, or tightly attached and remained bound to the material after rinsing. To determine the viability of loosely attached endothelial cells, we used a trypan blue (Sigma Chemical Co.) assay on the effluent from the material wash. To determine the viability of the tightly attached endothelial cells, we used calcein/ethidium (Molecular Probes, Inc) fluorescence directly on the material; this latter method was also used on the control cells on coverslips.

Light Microscopy.

Living endothelial cells in culture were viewed with fluorescence and phase contrast microscopy (Nikon E800) using 4×, 20× or 40× objectives. Trypan blue assay was assessed at 4× using a hemocytometer and appropriate counting statistics. Calcein/ethidium assay was assessed using fluorescence (Chroma filter sets) at 40×. Color images were taken with an Evolution QEi camera system (Media Cybernetic, and analyzed using Qimaging software.

Material Porosity-Hydraulic Conductivity.

The materials used with cells were evaluated for functional porosity by testing hydraulic conductivity of double thickness materials. A sheet of material was folded and gently placed between two gaskets that were sealed into a Teflon tubing line; the cross-sectional area of material open for water flow was 3.1×10⁻² cm². Water was pumped through the material by syringe pump (0.01-1 ml/min) and the balance pressure was determined. The slope of the balance pressure as a function of volume flow was used to estimate porosity, using the equation: flow=(hydraulic conductivity×surface area)×pressure.

CA Membranes.

In order to obtain continuous nanofibers, a minimum concentration of polymer solution is required. Below this, either droplets or combination of droplets and fibers will be observed. The reason for this is that under low concentration, the polymer chain entanglements might not be enough to make the jet stable so as to get the fibers [21]. FIG. 4 shows the SEM images of different compositions of CA solutions. Only droplets of polymers were seen in the SEM image of the material with concentration of 7.5%. With the increasing concentration of the polymer solution, fibers were observed under the concentration of 10.0%, though droplets still existed. Above the concentration of 11.0%, three-dimensional non-woven fibers were observed. The average diameters of the fibers obtained from electrospinning polymer solution increases with increasing concentration of the solution. However, when the concentration reaches 20.0%, the solution becomes too viscous to be electrospun. The diameters of fibers obtained varied from 100 nm to 1.2 μm while the average diameter was approximately 500 nm.

Endothelial Cell Growth on Membranes

Three materials were tested for their effect on endothelial cell viability: CA alone, CA+0.25% CT, CA+0.5% CT. Loose endothelial cells that could be easily washed off the matrix were tested with trypan blue, where dead cells appear blue and live cells appear clear. For all materials combined, 50-70% of the loosely attached cells were viable. CA alone (63±5%) was not different from 0.25% CT (65±4%), however, increasing CT concentration to 0.5% decreased viability by day 5 (58±3%*). Firmly adherent endothelial cells were tested using calcein/ethidium, where live cells appear green (due to calcein uptake and ethidium exclusion) and dead cells appear red (due to ethidium binding to nucleic acids). For all materials combined, total viability was ˜90%. CT had no effect on the number of firmly attached viable cells. For each material, endothelial cells appeared to initially attach to single fibers and spread across fibers (FIG. 5, left) and later form tubular-like structures (right). Apparent porosity of the membranes was determined by the pressure required to push a known volume of fluid through a double thickness of membrane. The hydraulic conductivity was 1.8×10⁻¹ cm² g⁻¹ for CA membranes and significantly less porous for CA+0.25% CT, 3.2×10⁻² cm² s g⁻¹ (5.6-fold difference). The higher concentration of CT (0.5%) was not different from 0.25% CT (3.0×10⁻² cm₂ s g⁻¹).

As discussed above the three-dimensional non-woven nanofibers membranes produced by the electrospinning process described herein can serve as the scaffolds for vascular endothelial cell growth. The structure of the membranes that were produced mimic the topography and porosity of natural extracellular matrix. The presence of CT significantly decreases apparent porosity, perhaps by strengthening the material. CT had no effect on the viability of cells that were tightly adhered to the membrane. Over time, cells appear to form tubular-like structures similar in appearance to vascular capillaries.

While the present invention has been described with reference to certain embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out the process of the invention but that the invention will include all embodiments falling within the scope of the appended claims.

### REFERENCES

[1] Stephen F. Badylak. The extracellular matrix as a scaffold for tissue reconstruction. Cell & development biology. 2002, 13, 377-383.
[2] Francois Berthiaume, Prabhas V. Moghe, Mehmet Toner, Martin L. Yarmush. Effect of extracellular matrix topology on cell structure, function, and physiological responsiveness: hepatocytes cultured in a sandwich configuration. Faseb Journal, 1996, 10, 1471-1484.
[3] Richard Skalak, C. Fred Fox. Tissue Engineering. UCLA Symposia on Molecular and Cellular Biology, New Series, Volume 107. Alan R. Liss, Inc., New York.
[4] Byung-Soo Kim, David J. Mooney. Development of biocompatible synthetic extracellular matrices for tissue engineering. Trends in Biotechnology, 2001, 16, 224-230.
[5] Linda G. Griffith. Emerging design principles in Biomaterials and scaffolds for tissue engineering. Reparative medicine: Growing tissues and organs annals of the New York Academy of Sciences 2002, 961, 83-95.
[6] Geoffrey M. Cooper. The Cell: A Molecular Approach. ASM Press, Washington, D.C. Sinauer Associates, Inc., Sunderland, Mass. Second Edition, 2000.
[7] Stephen F. Badylak. Extracellular matrix as a scaffold for tissue engineering in veterinary medicine: applications to soft tissue healing. Clinical techniques in equine practice, 2004, 3, 173-181.
[8] J. F. Kennedy, G. O. Phillips, D. J. Wedlock and P. A. Williams. Cellulose and its derivatives: chemistry, biochemistry and applications. Ellis Horwood Limited, 1985.
[9] Emilia Entcheva, Harold Bien, Lihong Yin, C.-Y. Chiung-Yin Chung, Melissa Farrell and Yordan Kostov. Functional cardiac cell constructs on cellulose-based scaffolding. Biomaterials, 2004, 25, 5753-5762.
[10] Matis Martson, Jouko Viljanto, Timo Hurme, Pekka Laippala, Pekka Saukko. Is cellulose sponge degradable or stable as implantation material? An in vivo subcutaneous study in the rat. Biomaterials, 1999, 20, 1989-1995.
[11] M. M. Demir, I. Yilgor, E. Yilgor, B. Erman. Electrospinning of polyurethane fibers. Polymer, 2002, 43, 3303-3309.
[12] Y. M. Shin, M. M. Hohman, M. P. Brenner, G. C. Rutledge. Experimental characterization of electrospinning: the electrically forced jet and instabilities. Polymer, 2001, 42, 9955-9967.
[13] M. Wang, H. Singh, T. A. Hatton, G. C. Rutledge. Field-responsive superparamagnetic composite nanofibers by electrospinning. Polymer, 2004, 45, 5505-5514.
[14] Jayesh Doshi, Darrell H. Reneker. Electrospinning process and application of electrospun fibers. Journal of Electrostatics, 1995, 35, 151-160.
[15] H. Fong, I. Chun, D. H. Reneker. Beaded nanofibers formed during electrospinning. Polymer, 1999, 40, 4585-4592.
[16] J. A. Frangos. Physical forces and the mammalian cell, 1993, Academic Press, San Diego, Calif.
[17] Jean D. Sipe. Tissue engineering and reparative medicine. Annals of the new york academy of sciences, 2002, 961, 1-9.
[18] J. M. Deitzel, J. Kleinmeyer, D. Harris, N. C. Beck Tan. The effect of processing variables on the morphology of electrospun nanofibers and textiles. Polymer 2001; 42:261-272
[19] Bloom and Fawcet: A Textbook of Histology. Fawcett D W, Editor, 11th Edition. WB Saunders Co, Philadelphia, 1986.
[20] Vernon R B, Gooden Md. New technologies in vitro for analysis of cell movement on or within collagen gels. Matrix Biology 2002; 21:661-669.
[21] Frame M D S, Chapman G B, Makino Y, Sarelius I H. Shear stress gradient over endothelial cells in a curved microchannel system. Biorheology, 1998; 35: 245-261.
[22] Kenawy E R, Layman J M, Watkins J R, Bowlin G L, Matthews J A, Simpson D G, Wnek G E. Electrospinning of poly (ethylene-co-vinyl alcohol) fibers. Biomaterials 2003; 24:907-913.

What is claimed is:

1. An extracellular matrix comprising a three-dimensional non-woven scaffold, said scaffold comprising one or more layers of one or more arrays of microfibers, wherein said three-dimensional non-woven scaffold is arranged to mimic a configuration of natural extracellular matrix (ECM).

2. The extracellular matrix of claim 1 wherein an electrospinning process is used to produce said extracellular matrix.

3. The extracellular matrix of claim 2 wherein said one or more arrays of microfibers contain cellulose acetate (CA).

4. The extracellular matrix of claim 3 wherein said extracellular matrix further comprises carbon nanotubes (CT).

5. The extracellular matrix of claim 1 wherein the microfibers have a diameter of about 0.5 μm to about 10 μm.

6. The extracellular matrix of claim 5 wherein spacing of adjacent microfibers in one or more layers of one or more arrays is about 10 micrometers to about 100 micrometers.

7. The extracellular matrix of claim 6 wherein said spacing of adjacent microfibers in one or more layers of one or more arrays is about 30 micrometers to about 70 micrometers.

8. The extracellular matrix of claim 6 wherein said microfiber scaffold is coated with a cell adhesion-enhancing agent.

9. The extracellular matrix of claim 8 wherein said cell adhesion-enhancing agent is selected from the group consisting of collagen, laminin, and fibronectin.

10. The extracellular matrix of claim 8 further comprising cells cultured on said scaffold to form a target tissue substitute.

11. The extracellular matrix of claim 10 wherein said target tissue is an arterial blood vessel, wherein an array of microfibers is arranged to mimic a configuration of elastin in a medial layer of an arterial blood vessel.

12. The extracellular matrix of claim 8 further comprising cells cultured on said microfiber scaffold to form a blood vessel substitute.

13. The extracellular matrix of claim 8 further comprising muscle cells cultured on said microfiber scaffold to form muscle tissue.

14. The extracellular matrix of claim 8 wherein said muscle cells further comprise endothelial cells cultured on said microfiber scaffold to form endothelial tissue.

15. The extracellular matrix of claim 13 wherein the array of microfibers is arranged so as to mimic a configuration of smooth muscle fibers in muscle tissue.

16. The extracellular matrix of claim 13 wherein said muscle tissue is skeletal muscle tissue or cardiac muscle tissue.

17. The extracellular matrix of claim 10 wherein said target tissue substitute is endothelia tissue, wherein said microfiber scaffold comprises at least three layers of microfibers, wherein a first array of microfibers is arranged to mimic a dense layer of the endothelia tissue, wherein a second array of microfibers is arranged to mimic a cellular layer of the endothelia tissue, and wherein a third layer of microfibers is arranged to mimic a fibrous layer of the endothelia tissue.

18. The extracellular matrix of claim 10 wherein said microfiber scaffold mimics a urinary bladder matrix (UBM).

19. The extracellular matrix of claim 19 wherein said microfiber scaffold further comprises a keratinocyte growth substrate.

20. The extracellular matrix of claim 10, wherein said target tissue substitute is cartilage tissue, and wherein said microfiber scaffold is arranged to mimic a configuration of collagen fibers in fibrous cartilage tissue.

21. The extracellular matrix of claim 20 further comprising cells cultured on said scaffold to form a cartilage substitute.

22. A method for synthesizing an extracellular matrix comprising a three-dimensional non-woven scaffold, said method comprising:
(a) dissolving at least one polymer in a biocompatible solvent to produce an electrospinning polymer solution;
(b) subjecting the electrospinning polymer solution of step (a) to an electric field between about 5 kV and about 20 kV at a flow rate between about 10 l/min and about 200 l/min to produce single layer mats of variable morphologies.

23. The method for synthesizing an extracellular matrix of claim 22 wherein said at least one polymer is cellulose acetate (CA).

24. The method for synthesizing an extracellular matrix of claim 23 further comprising adding carbon nanotubes (CT) to solution (a) to produce a CA-CT electrospinning solution.

25. The method for synthesizing an extracellular matrix of claim 24 wherein step (b) is performed on the CA-CT electrospinning solution prior to precipitation of said carbon nanotubes (CT) from solution.

26. The method for synthesizing an extracellular matrix of claim 25 wherein at least three single layer mats of variable morphologies are produced so as to mimic a configuration of one or more structural elements in a target tissue.

27. The method for synthesizing an extracellular matrix of claim 26 wherein porosity within said non-woven scaffold is controlled by electrostatically controlled templating that is not disruptive to the electrospinning process.

28. The method for synthesizing an extracellular matrix of claim 27 wherein said process of electrostatically controlled templating comprises providing a removable copper wire template mimicking the desired porosity prior to said electrospinning process and removing said removable copper wire template from said non-woven scaffold once said electrospinning process is substantially completed.

29. The method for synthesizing an extracellular matrix of claim 26 wherein a removable copper wire template mimicking the desired porosity is provided between at least two adjacent layers of said at least three single layer mats prior to electrospinning to induce the electrospinning process to produce a layer that mimics the copper wire template and removing the copper wire template once the electrospinning process is completed.

30. The method for synthesizing an extracellular matrix of claim 22 further comprising culturing cells on said non-woven scaffold to produce a target tissue substitute.

31. The method of claim 30 wherein the target tissue is an arterial blood vessel, wherein an array of microfibers is designed to mimic the configuration of elastin in a medial layer of an arterial blood vessel and wherein cells are cultured on the non-woven scaffold to form a blood vessel substitute.

32. The method of claim 31 wherein said cells are selected from the group consisting of smooth muscle cells, endothelial cells and mixtures thereof.

33. The method of claim 22 wherein the non-woven scaffold comprises about 2 to about 25 layers.

34. The method of claim 30 wherein said target tissue substitute is selected from the group consisting of skeletal muscle tissue, cardiac muscle tissue, fibroblasts, cartilage, heart valve tissue, liver tissue, kidney tissue and mixtures thereof.
